(19) United States
(12) Patent Application Publication
(10) Pub. No.: US 2023/0019597 A1
(43) Pub. Date: Jan. 19, 2023

(54) APPARATUS AND METHOD FOR RETREIVING INFORMATION FROM A COMPUTER SYSTEM FOR STORAGE IN A CLOUD ENVIRONMENT

(71) Applicant: Sorna Corporation, Eagan, MN (US)

(72) Inventor: Cyrus Kurosh SAMARI, Ladera Ranch, CA (US)

(21) Appl. No.: 17/950,477

(22) Filed: Sep. 22, 2022

Related U.S. Application Data
(63) Continuation of application No. 14/091,806, filed on Nov. 27, 2013, now abandoned.
(60) Provisional application No. 61/730,135, filed on Nov. 27, 2012.

(51) Int. Cl. G16H 10/60; G16H 30/20

(52) U.S. Cl. CPC G16H 10/60; G16H 30/20

(57) ABSTRACT

A module for retrieving information from a computer system and storing information in a cloud for retrieval by authorized persons. The module can also store the gathered or retrieved information onto media. The module can be hardware or software or combination of hardware and software.

### CROSS-REFERENCE TO RELATED APPLICATION

This application is a Continuation in Part of U.S. Non-Provisional application Ser. No. 14/091,806 filed Nov. 27, 2013, entitled “APPARATUS AND METHOD FOR RETRIEVING INFORMATION FROM A COMPUTER SYSTEM FOR STORAGE IN A CLOUD ENVIRONMENT” which claims the benefit of U.S. Provisional Application No. 61/730,135 filed Nov. 27, 2012, entitled “APPARATUS AND METHOD FOR RETRIEVING INFORMATION FROM A COMPUTER SYSTEM FOR STORAGE IN A CLOUD ENVIRONMENT” which is incorporated herein by reference. A claim of priority is made.

### BACKGROUND AND ENVIRONMENT

A hospital has many departments that generate medical images and data such as physicians notes, lab reports, patient information, patient history, prescriptions, and the like.

There are family physicians, specialist physicians (orthopedics, neurologists, cardiologists, and the like) surgeons, and other care givers who need access to part or all of the data a hospital has. This includes image data. These caregivers also produce similar medical data (X-ray, notes, blood pressure, temperature, medical history, allergy, and the like). Many times these care givers refer the patient to the hospital for imaging or other procedures such as lab work, endoscopy, surgery, etc.

As a result of activities of taking care of a patient, care givers need access to medical images and data from multiple locations (their office, at the hospital, at home, in the surgical suite, at patient bed side, etc.). So it is critical for patient images, data and all associated medical records be available anywhere. Of course security, privacy, and audit trail concerns are key elements in the medical environment as well.

The practice of medicine currently includes sending a patient to various institutions for obtaining images for use in the practice of medicine. A doctor may refer a patient to a third-party provider, such as a hospital or imaging clinic, to get a needed image for a diagnosis. These third-party providers then send an image and/or other medical information back to the referring physician. The problem exists in that a successful third-party provider will have a number of referring physicians and other referring institutions. Each of these institutions may have a different cloud provider. Needless to say, even if some of the referring parties have a common cloud provider, the third-party provider still has to deal with and send medical information through a number of different cloud providers to get the information back to the referring physicians or referring hospitals. Each of the cloud providers that the third-party deals with can have a different API (application program interface). Therefore sending information, such as medical information, back to referring parties through a series of cloud providers requires that the sender or third-party provider has the ability to obtain the correct API for a particular cloud. As can be imagined, this can be both complex and difficult. Especially considering that there are a number of cloud providers used by the referring parties. The third-party provider has to keep track of the referring party, the cloud that the referring party uses, and the API needed to provide connectivity to that particular cloud.

### SUMMARY OF THE INVENTION

As of late, “cloud” based solutions (e.g. LifeImage, emix, Merge honeycomb, DICOMgrid, etc) are becoming popular and increasingly secure with extensive permission and audit trails. Cloud solutions usually have an application program interface (API) available to third parties for connectivity. Standards are also in place for the medical industry (such as HL-7, DICOM, IHE, and the like) that describe secure sharing of medical images and information.

This invention teaches a method and apparatus for retrieving and/or receiving medical information from a system and storing in or forwarding the medical data to cloud storage so that any authorized user can obtain or access the medical information. In one embodiment the invention is a module that is part of an image sharing server. The module can be a combination of hardware and software, or just software or just hardware. The image sharing server is also capable of producing images or medical information on various types of media, including portable media, such as a CD-ROM, floppy disk, DVD, memory stick or the like. The image sharing server is also capable of producing information, such as images or medical information on all types of media, including nonportable media such as memory within a computer or network.

The module, in one embodiment, includes a set of instructions for causing a processor associated with the computer to gather or otherwise receive information from a network and either place the resulting gathered information onto a media or present the same or portions of the medical information to a cloud-based storage provider via the Internet or other network. In some embodiments, the module can also directly share information, such as medical information, between a first location and a second location without passing through the cloud provider. The module is named SAM. This invention concerns itself more with connectivity to these APIs and standards, from a sending and receiving site view point rather than dealing with the inner workings of a particular cloud transport provider or standards descriptions.

The module simplifies act of sending results between parties. For example, a third party provider such as a hospital merely has to choose the referring party to whom to send the results. Rather than having to connect to each API for a particular cloud transport system, the SAM module keeps track of a referral source, as well as the referral source's method of sharing/connectivity such as CD/DVD/BD, other portable storage device (e.g. memory stick), cloud provider, or direct secure share and the like.

### BRIEF DESCRIPTION OF THE DRAWINGS

FIG. 1 is a schematic view of network connected to the Internet and the cloud that includes an image sharing server capable of placing information in the cloud for storage on and retrieval from the cloud, according to an example embodiment.

FIG. 2 is a schematic view of a computer system, according to an example embodiment.

FIG. 3 is a schematic drawing of a machine readable medium that includes an instruction set, according to an example embodiment.

FIG. 4 is a schematic view of a system including a plurality of doctors linked via the cloud to a number of third party providers.

FIG. 5 is a schematic view of a doctors system which is linked to a plurality of third party providers, according to an example embodiment.

FIG. 6 is a schematic drawing of a SAM configured as a workstation, according to an example embodiment.

FIG. 7 is a schematic drawing of a SAM configured as a sharing server, according to an example embodiment.

FIG. 8 is a schematic drawing of a computing system having a SAM configured within a system, according to an example embodiment.

FIG. 9 is a screen shot of a SAM user interface for configuring a server, according to an example embodiment.

FIG. 10 is a screen shot of a SAM user interface which is used to define the label for a type of media, according to an example embodiment.

FIG. 11 is a screen shot of a SAM user interface which is used to define a query for a patient's health information, according to an example embodiment.

### DETAILED DESCRIPTION OF THE INVENTION

F or the lack of a better terminology, this invention will be referred to as SAM.

### Definitions

In this application the following terms are defined.

A. Medical information is any information that includes patient health information (PH I). This includes radiology from any known modality or other source, physicians notes, nurses notes, bitmaps, JPEG's, pictures, lab results, lab reports, Pathology images, information, and reports, information generated by other medical devices (e.g. blood pressure, digital thermometer, . . . ), information form Hospital Information Systems (HIS), information from Radiology Information Systems (RIS), Cardiology systems, Electronic Medical Records (EMR) systems and the like. Medical information can be in DICOM or HL-7 or any other format.
B. Retrieve is seeking information and obtaining information that was asked for. The information can include medical information.
C. Receive is obtaining information, such as medical information, that was not necessarily requested.
D. Direct send is routing patient information from a first storage area to a second storage area without passing the information, such as medical information, through a cloud provider. Obviously the information has to move through some type of a network, private, public or both.
E. Secure send is sending patient information securely by direct send, via cloud provider, public network, virtual private network or other route.

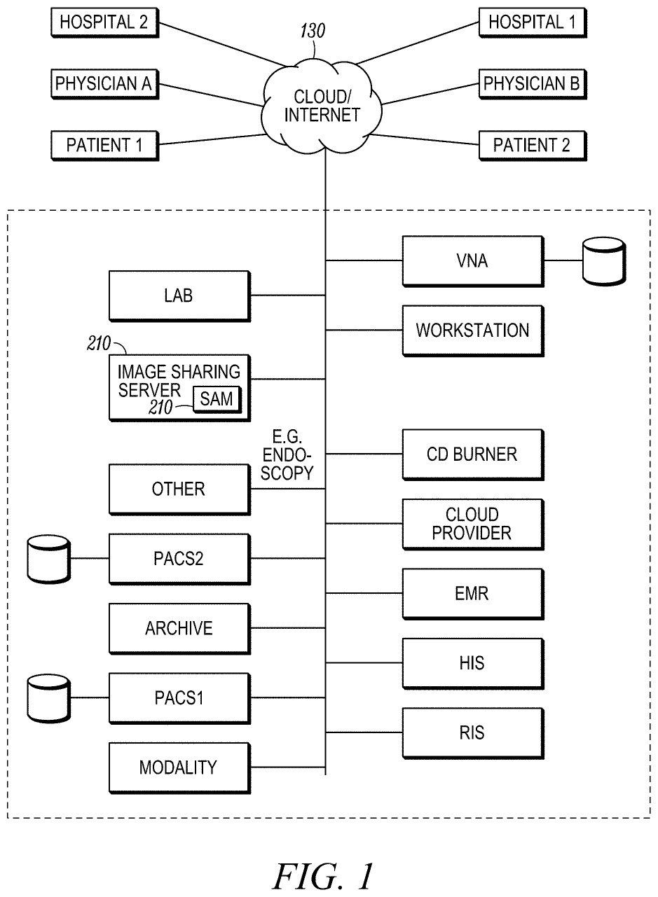

FIG. 1 is a schematic view of network 101 connected to the Internet and the cloud 130, according to an example embodiment. On a network 101 is an image sharing server 200 capable of placing information, such as medical information, in the cloud 130 for storage on and retrieval from the cloud 130, according to an example embodiment. The image sharing server 200 includes a module 210 that is an apparatus that performs various methods for retrieving and/or receiving medical information from a system or network 101. The module 210 is called or referred to as SAM. Attached to the network 101, in the example shown in FIG. 1, are various sources of medical information, such as data and other information. In this particular example, the various sources of medical information and other information include sources associated with a medical network that might be found in the hospital, research center, or in an office or clinic for practicing medicine. It is contemplated that the invention is not limited to such a network but can actually be used on any similar network.

In one embodiment, the module 210 is part of an image sharing server 200. The module can be a combination of hardware and software, or just software or just hardware. The image sharing server 200 is also capable of producing images on various types of media, including portable media such as a CD-ROM, floppy disk, DVD, memory stick or the like. The image sharing server 200 is also capable of storing medical data in an associated memory or in a memory of another device. The module 210, in one embodiment, includes a set of instructions for causing a processor associated with the computer, such as a computer housed within the image sharing server 200, to gather or otherwise receive information from a network and either place the resulting gathered information onto a media or present the same or part to a cloud-based storage provider via the Internet or other network. The module 200 could also be placed in another computer on the network 101. In another embodiment, the module 210 could be placed on a dedicated computer attached to the network 101. In still another embodiment, the module 210 could be placed in a media burner capable of automatically producing media from information on a medical network. Still another embodiment, the module 210 could be placed in a server that is configured to have a single client or server that is configured to have multiple clients.

If the module 210 stores the gathered or retrieved medical information, security measures will be in place to limit access to the information so that only an authorized user can access the medical information, such as medical images or data. In the example shown in FIG. 1, a pair of hospitals, a pair of physicians, and a pair of patients are the authorized users that can access the information stored on the cloud. The cloud includes storage. The cloud can also include programs. The cloud is accessed via the Internet. The cloud allows an authorized user to gain access to the information stored thereon by a patient or physician or hospital at any place where there may be an Internet connection. In other words, this provides the patient, physician or hospital to conveniently have access to information or data. Of course a physician or hospital may also have more permanent records and may store this information on site at either the hospital or a physician's clinic. In other words the module 210, which is the software/system/solution can be used to store the information on the cloud and produce information on a media so that it can be permanently stored in a hospital or clinic or the like.

For the sake of simplicity, the software/system/solution referred to as SAM and denoted by module 210 will be referred to as SAM or a software/system/solution in the remaining text. There are typically two methods of interface with a given software/system/solution. First is user-initiated and second is automatic. This invention deals with both of these methods as well as a combination of the two.

With an automated method, the workflow configuration of the system initiates and completes the process of sharing. For example, when a radiology report is generated (usually because a Radiologist has read an X-ray, MRI, CT, US, . . . and dictated a report) the Radiology Information System (or another application responsible for these tasks) sends an HL-7 message to all devices/application/systems configured to receive these messages. SAM is one such application/appliance.

It is important to note that even though the concept of an HL-7 message is discussed, there are many other methods a report can be received by SAM (e.g., a DICOM send from any node on the network, and XML or other format message via an API or network interface, a hot folder, . . . ). Once SAM receives this (ese) object(s), it creates a new job/task/operation and continues to perform a series of steps that form a method:
1. Scan/parse the data and extract/get important physician, patient and study information. Patient information is usually Patient name, ID, and DOB. Physician is the referring physician that requested the study to be made which is usually name, some type of ID, facility (physician's office or institution) name and/or id, Address, . . . Study information is typically a unique Accession number, Date, time, description, body part, modality, and other information or medical information.
2. SAM can be configured to modify the format of the report for example to a DICOM Structured Report (SR), or HTML, XML, Text, RFT, Word, . . . or anything else that may be required. It is contemplated that other formats for reports may be required in the future. It is contemplated that SAM can be configured to these future formats.
3. Next SAM determines the Radiology study (images) associated with this report. Typically, an Accession # is the common field between the Radiology studies and the reports. It is supposed to be unique. Hence SAM will perform a query of the archive, such as a PACS archive, or a Vendor Neutral Archive (VNA) storing more than just Radiology images. The vendor neutral archive can include but is not limited to cardiology, endoscopy, ophthalmology, pathology, dental, and other medical information sent to it for storage. SAM performs a query of the archive using the best data (patient, study and physician data or any other medical information) available to it from the report to ensure finding the proper study(ies). The query can be based on medical standards (DICOM query) or any other API made available by the archive. SAM will analyze the result of the query and determine which study(ies) to retrieve from the archive. It is critical to note that usually data format varies from typical reports (HL-7) when compared to data in images (DICOM). For example, an HL-7 message will have the patient name as “Public, John Q”. The same name in a DICOM object is stored as “Public \hat{ }John\hat{ }Quincy\hat{ }Mr”. Same types of differences exist in date format. SAM is intelligent enough to deal with these differences when associating a study with a report.
4. SAM will next keep track of the study(ies) it will be retrieving so they can be identified once received and associated with this task/job/operation. Of course received report(s) are also part of this task/job/operation.
5. SAM will next request the study(ies) from the archive. This can be done via the DICOM standard move request or any API or available to SAM. It is contemplated that SAM will be configured to deal with future standards. The archive will then send the requested studies to SAM via the DICOM standard store operation or any API or future standard.
6. SAM will receive this(ese) study(ies), determine which task/job/operation they belong to and associate them with it. Once SAM has received the(all) associated study(ies) with the report(s), it will then proceed to perform the tasks it is configured to.
7. Sam, if so configured, can search for “relevant priors”. “relevant priors” can be configured to be any exam within a given time frame (3 month, 1 year, 5 years, forever, . . . ) and/or specific (X-ray, US, MR, CT, . . . ) or any modality, and/or matching body part (crane, chest, abdomen, right knee, . . . ) or any body part. The archive(s) in which SAM is to look for such priors can also be configured. SAM can also be configured to look for any reports or information for such priors in one or more devices. If configured to do so, SAM will search and retrieve relevant priors from all configured devices (archives(s), report repository(ies), EMR systems, RIS systems, databases, . . . ). Depending on the type of system (and its interface) SAM is configure to search, SAM might have to retrieve more than the configured relevant priors in order to properly determine what information matches the “relevant priors” configured. Once SAM has gathered all the “relevant priors” and associated information, it will go to the next step to continue processing.
8. Tasks that SAM may be configure to do are: burn a DISC or multiple DISCs (according to DICOM/IHE standards) utilizing automated CD/DVD/BD burners and printers, send to the appropriate cloud configured, perform a direct send (secure or over a secure network, or both) to the referring/requesting physician or institution, send a copy to the patient (perhaps minus the report, if so configured), create a non-diagnostic copy (e.g. lossy images minus reports, if so configured) to share with patient.
9. If so configured, the non-diagnostic patient copy can be created in many formats. For example, HTML page(s), MPEG, AVI or other video formats out of a multi-image (CT, MRI, . . . ) or multi-frame (Ultrasound, endoscopy, cardiology, multi-frame MRI, . . . ) study or convert a multi-frame into simple consecutive images.
10. SAM can also be configured to automatically exclude certain series (one or more) from a study. Such series usually contain scanned documents (order form, requisition form, . . . ) or other forms or documents that were added to the study or created as part of the original study. SAM will exclude based on series number, description, or other fields as configured.
11. Should SAM be unable to complete any of the tasks it is configured to, it has the ability to pause that particular task or the group of associated tasks, and ask for an operator intervention. The operator can then choose to re-start the tasks, make changes to the tasks, or cancel. For example, SAM may be unable to find a study(ies) associated with the report(s), or the transfer of the study(ies) from the archive may fail. Also the sending of the study(ies) and report(s) to the cloud may fail for a variety of reasons, and many other possible errors may occur. SAM may also automatically cancel a failing job—if configured so. SAM also may be configured to display a status of various tasks. For example, if certain tasks are incomplete it can show that on a user interface so that appropriate action can be taken, if need be.

Another method of automated operation is when a study(ies) is(are) sent to SAM from the PACS or modality or any other device based on its workflow (configured to automatically send certain studies to SAM based on configured rules). Very similar operations to the above then occurs. Once the study(ies) has been received, SAM creates a new job/task/operation and proceed with the following steps:
1. SAM will Scan/parse the data and extract/get important physician, patient and study information. Patient information is usually Patient name, ID, and DOB. Physician is the referring physician that requested the study to be made which is usually name, some type of ID, facility (physician's office or institution) name and/or id, Address, and the like. Study information is typically a unique Accession number, Date, time, description, body part, modality, and the like. The study information can include information parsed from medical information, such as a DICOM file.
2. SAM, if so configured, will attempt to search for and get the associated report(s) for the study. This function depends on the hospital PACS, RIS, HIS, EMR, and other systems. SAM can use HL-7, DICOM, database interface, HTML call, custom API, or any other method as required by the hospital or healthcare system's infrastructure. SAM will typically interface with its reports module which will in turn be setup to work with one or more interfaces to the healthcare system's infrastructure. SAM uses information gathered in step 1 above to determine what fields to use to query and how to associate the received data/information with the received study(ies). The process is very similar to the ones described above.
3. Sam, if so configured, can search for “relevant priors”. “relevant” can be configured to be any exam within a given time frame (3 month, 1 year, 5 years, forever, . . . ) and/or specific (X-ray, US, MR, CT, . . . ) or any modality, and/or matching body part (crane, chest, abdomen, right knee, . . . ) or any body part. The archive(s) in which SAM is to look for such priors can also be configured. SAM can also be configured to look for any reports or information for such priors in one or more devices. If configured to do so, SAM will search and retrieve relevant priors from all configured devices (archives(s), report repository(ies), EMR systems, RIS systems, databases, . . . ). Depending on the type of system (and its interface) SAM is configure to search, SAM might have to retrieve more than the configured relevant priors in order to properly determine what information matches the “relevant priors” configured. Once SAM has gathered all the “relevant priors” and associated information, it will go to the next step to continue processing.
4. Once SAM has received the study(ies) and the associated report(s) and/or information and it has gathered any relevant priors and associated information, it can then start to process the tasks it is configured to do—just as described above.

Another method of automated operation for SAM, as a modification/configurable alternative to the above is for SAM to receive the study(ies) and if the report(s) is(are) not available, SAM will wait for it (them) to become available and then proceed with its tasks to perform. This is a very typical situation where medical information needs to be interpreted. The medical information becomes available initially and the interpretation or report follows after an image or other diagnostic medical information becomes available.

There are two ways SAM can wait for reports, depending on the Healthcare institution infrastructure and SAM's configuration:
1. Once a report(s) is available, it is automatically sent to SAM, where it is received, scanned/parsed, and then associated with the awaiting study(ies) and then SAM performs the tasks associated with the job/operation—as described above.
2. SAM, if so configured, will periodically query the configured system awaiting a report(s). Once appropriate report(s) has(ave) been retrieved, it will continue to process the job/operation as described above.

In the above cases, SAM can also be configured to include “relevant priors”.

A combination of user-initiated and automated operation is when an operator initiates sending a study(ies) or report(s) to SAM from a workstation in the healthcare institution infrastructure. The same operations as above will then be performed by SAM to share the study(ies) and report(s)—as configured.

When submitting job(s) to the autoloader for recording and printing CD/DVD/BD DISCs, SAM is quite smart in function. It can be configured to force the selection of one media type by the autoloader. It will then make smart decisions based on configuration to split a study(ies) among two or more DISCs. When splitting stuy(ies), SAM can split at patient, study, series or instance (single file) level—based on configuration. It can also be configured to analyze the data and choose whether to split at patient, study, series or instance level. SAM, depending on the configuration and/or the capability of the autoloader chooses the appropriate DISC (CD/DVD/BD) that will fit the data. SAM is also capable of encrypting the content of the DISC with a password (if so configured). The password can be one of fields of the data/images received by SAM or user-provided—depending on configuration.

When printing the data on the DISC, patient, study, physician, creating institution information (name logo, . . . ), receiving entity, number of DISCs (on 1 of 3), DISC type (e.g. CD/DVD/BD, . . . ) date and time of creation, unique serial number, whether it is encrypted).

SAM tracks all of its functions includes the data it has shared by making DISCs or sending to cloud or direct sharing in a database. Details of the study(ies) images, data, reports, sharing methods, and all other details are included in this database.

Another method of operation for SAM is user-initiated. SAM's user interface allows users all over the healthcare institution to login (if so configured—perhaps using single sign-on utilizing LPAD, AD or other protocols). Once logged in, the user has many functions at their disposal.

The user interface for SAM, allows functions to become “visible” depending on the user privileges. Functions available thru the SAM user interface include (but not limited to:
A dashboard for overall view of the SAM status
Monitoring the status of each job/task
Monitoring the status of each autoloader configured in SAM
Providing an interface for querying one or multiple PACS or VNA archives, displaying the results and allowing the user to select one or more patients, studies, series or instances.
Once selected, the user can then choose a multitude of options for burning and or sharing (cloud or direct) the data. For example the user can select to burn a DISC to autoloader 1 and send to Referring Physician Office via cloud B. User has the choice of selecting:
   Media Type
   Media split method
   Media template (viewer, label format to be printed on the DISC)
   Option to include reports
   Option to anonymize the data/images
   Option to choose how many DISCs to produce and on which autoloaders
   Option to choose who the DISCs are provided to (SAM will retain this information in its tracking database)
   Option to choose which institution/physician the data is sent via which cloud or direct sharing
   Option to choose if the data is provided to the patient, in what format, and using which cloud or method
   Option to add other material to the data/images (scanning documents and/or film, including popular file formats: word, txt, excel, html, xml, JPEG, AVI, MPEG, . . . )
   Option to add the other material as DICOM
Canceling jobs/operations or responding to SAM's error conditions
Running reports or querying the SAM tracking database
Configuring users and their privileges
Configuring the SAM system

Once the user selections have been made and submitted, SAM will take over, retrieve the stuy(ies), add the optional scans/documents/files, get the optional report(s) or wait for the report as described above, submit the job(s) to the autoloader(s)—if requested by user, send to cloud(s) or direct—if requested by user, encrypt the data—if requested by the user, and send direct—if so requested by the user.

The methods described above can be executed on a computer or computing device to form a specialized machine. In one embodiment, the specialized machine is a SAM that is an apparatus for retrieving information from a system and storing it at a location in the cloud for retrieval by authorized personnel according to the embodiments discussed above.

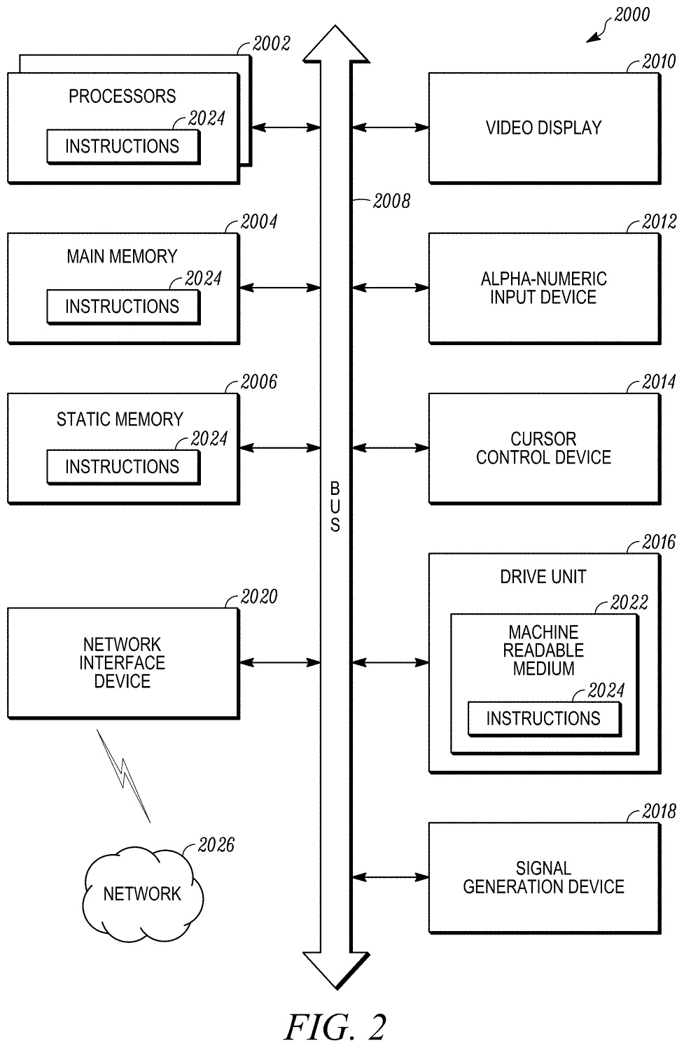

FIG. 2 shows a diagrammatic representation of a computing device for a machine in the example electronic form of a computer system 2000, within which a set of instructions for causing the machine to perform any one or more of the methodologies discussed herein can be executed or is adapted to include the apparatus for generating radiation reports as described herein. In various example embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a personal computer (PC), a tablet PC, a set-top box (STB), a Personal Digital Assistant (PDA), a cellular telephone, a portable music player (e.g., a portable hard drive audio device such as a Moving Picture Experts Group Audio Layer 3 (MP3) player, a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 2000 includes a processor or multiple processors 2002 (e.g., a central processing unit (CPU), a graphics processing unit (GPU), arithmetic logic unit or all), and a main memory 2004 and a static memory 2006, which communicate with each other via a bus 2008. The computer system 2000 can further include a video display unit 2010 (e.g., a liquid crystal display (LCD) or a cathode ray tube (CRT)). The computer system 2000 also includes an alphanumeric input device 2012 (e.g., a keyboard), a cursor control device 2014 (e.g., a mouse), a disk drive unit 2016, a signal generation device 2018 (e.g., a speaker) and a network interface device 2020.

The disk drive unit 2016 includes a computer-readable medium 2022 on which is stored one or more sets of instructions and data structures (e.g., instructions 2024) embodying or utilized by any one or more of the methodologies or functions described herein. The instructions 2024 can also reside, completely or at least partially, within the main memory 2004 and/or within the processors 2002 during execution thereof by the computer system 2000. The main memory 2004 and the processors 2002 also constitute machine-readable media.

The instructions 2024 can further be transmitted or received over a network 2026 via the network interface device 2020 utilizing any one of a number of well-known transfer protocols (e.g., Hyper Text Transfer Protocol (HTTP), CAN, Serial, or Modbus).

While the computer-readable medium 2022 is shown in an example embodiment to be a single medium, the term “computer-readable medium” should be taken to include a single medium or multiple media (e.g., a centralized or distributed database, and/or associated caches and servers) that store the one or more sets of instructions and provide the instructions in a computer readable form. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, tangible forms and signals that can be read or sensed by a computer. Such media can also include, without limitation, hard disks, floppy disks, flash memory cards, digital video disks, random access memory (RAMs), read only memory (ROMs), and the like.

The example embodiments described herein can be implemented in an operating environment comprising computer-executable instructions (e.g., software) installed on a computer, in hardware, or in a combination of software and hardware. Modules as used herein can be hardware or hardware including circuitry to execute instructions. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software programs for implementing the present method(s) can be written in any number of suitable programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Perl, UNIX Shell, Visual Basic or Visual Basic Script, Virtual Reality Markup Language (VRML), ColdFusion™ or other compilers, assemblers, interpreters or other computer languages or platforms.

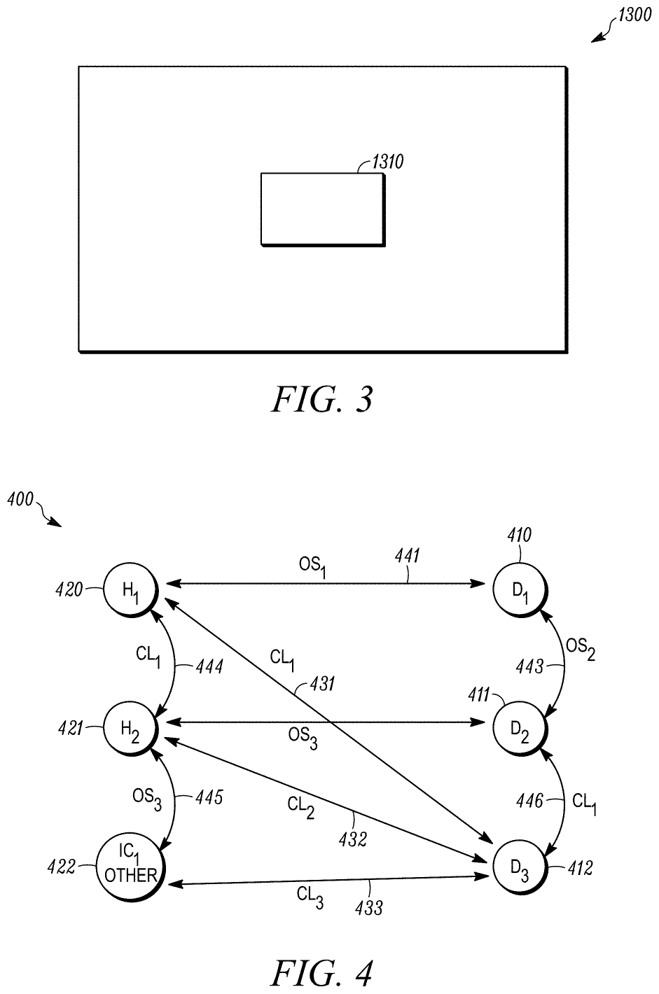

FIG. 3 is a schematic drawing of a machine readable medium 1300 that includes an instruction set 1310, according to an example embodiment. The machine-readable medium 1300 that provides instructions 1310 that, when executed by a machine, cause the machine to perform operations including eliciting and receiving an input to identify a selected investment, and eliciting and receiving an initial offering price for the investment. The machine readable medium 1300 also includes instructions that, when executed by a machine, cause the machine to perform operations that include receiving an input related to prompt displayed on a recycling container, identifying a marketing opportunity associated with the prompt, identifying the source of the received input, and sending the marketing opportunity to the source.

The present disclosure refers to instructions that are received at a memory system. Instructions can include an operational command, e.g., read, write, erase, refresh, etc.; an address at which an operational command should be performed; and the data, if any, associated with a command. The instructions can also include error correction data.

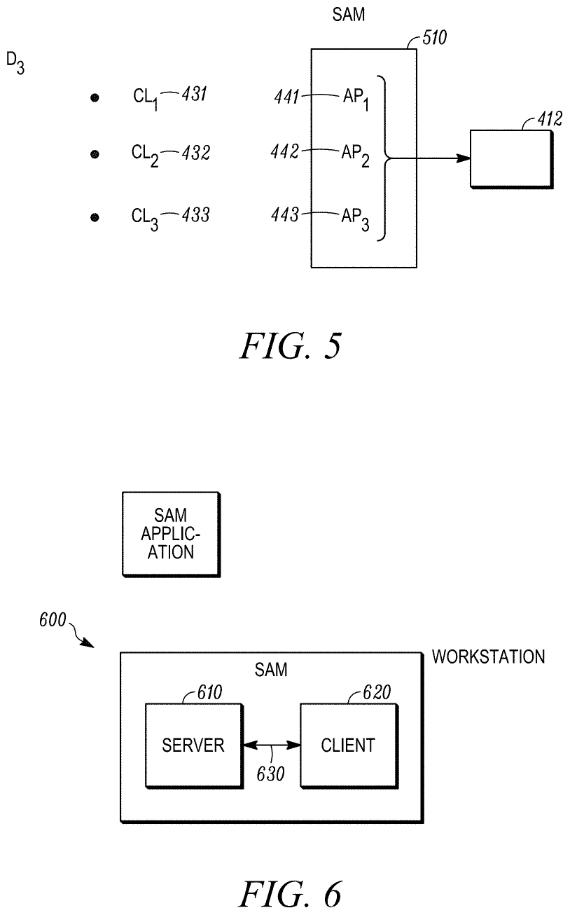

FIG. 4 is a schematic view of a system 400 including a plurality of doctors 410, 411, 412 linked via various links to a number of third party providers 420, 421, 422. The doctors 410, 411, 412 are linked to the third-party providers, such as hospital₁ 420 hospitals 421, and Imaging Center(s)₁ 422 via cloud provider connections 431, 432, 433 and via direct send connections 441, 442. Doctor₁ 410 is also connected to Doctor₂ 411 via direct send connections 443. Hospital1 420 is connected to Hospital₂ 421 via Cloud Provider₁. Hospital₂ 421 is connected to Imaging Center₁ 422 via direct Send 445. Doctor₂ 411 is connected to Doctor₃ via Cloud Provider₁ 446. It should be noted that any of the connected parties may use a cloud connection from a cloud provider to send to more than one place. In the example embodiment shown, hospital₁ uses cloud₁ to connect to hospitals and to Doctor₃. Hospital₁ can use the same API and same connections to connect to the cloud provider to send to more than one receiver. Any of these connections can be secure. For example, the interconnection 441 between hospital₁ 420 and doctor₁ 410 can be a secure direct send connection. These connections can be on a secure private network, a VPN, or using the public network (internet). It is also contemplated that any of the cloud connections are cloud transport mechanisms 431, 432, 433 could also be secure. A SAM device can be positioned in any one of the doctors' offices or clinical offices 410, 411, 412 or can be positioned or part of the third-party providers 420, 421, 422. A SAM device includes a software instruction set which allows for configuration of the server in the system in which it is placed. This SAM device includes a connection module which eases the management of connections in either sending or receiving information, such as medical information. This is further detailed in FIG. 5.

FIG. 5 is a schematic view of a doctors system 500 which is linked to a plurality of third party providers 420, 421, 422, and doctor3 412 according to an example embodiment. More specifically, FIG. 5 shows the doctor's office or physician's clinic 412 connected to the third-party providers 420, 421, 422 and Doctor3 412 by way of three cloud 431, 432, 433 connections. Each of the clouds 431, 432, 433, 446 includes an API 441, 442, 443. API 441 associated with cloud 431, API 442 is associated with cloud 432, and API 443 is associated with cloud 433. The APIs 441, 442, 443 are typically different for each cloud 441, 442, 443. The SAM device 510 includes software and hardware which allows the doctors system 412 to connect with each of the clouds 441, 442, 443 without having to manually link up via the APIs 441, 442, 443. In other words, the SAM device 510 includes memory which recalls the APIs 441, 442, 443 for each of the clouds 431, 432, 433 third-party providers 420, 421, 422. When sending medical information, the SAM device 510 associates the recipient with a particular cloud in a particular API associated with that cloud. Therefore the user merely has to upload the device to the recipient and can avoid having to select a particular cloud transport and the API associated there with. The SAM device remembers the API requirements as well as the cloud associated with a particular recipient. This results in a simplified sending process for sending medical information from the doctors' office to a third-party recipient or for receiving medical information from a third-party recipient.

FIG. 6 is a schematic drawing of a SAM 600 configured as a workstation, according to an example embodiment. Workstation 600 has a server 610 and a client 620. The server 610 and the client 620 are connected by a link 630.

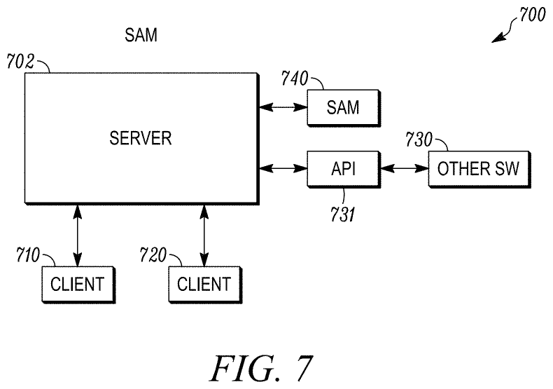

FIG. 7 is a schematic drawing of a system 700 configured as a sharing server, according to an example embodiment. The system 700 includes a first client 710 and the second client 720 and another server 730. The system 700 also includes a server 702. The first client 710 and second client 720 are internal clients for hospital or third party provider. The other server 730 can be an external connection or can be another internal connection. For purposes of this discussion, the other server 730 is connected externally. The connection includes an API 731 for the other server. The system 700 also includes a SAM device 740 which interfaces with the API 731. The API 731 will prevent unauthorized access to the other server 730. In this way the other server or the owner of the other server 730 can limit the amount of access to programming or other data within the other server 730. The SAM device 740 includes a memory which is relied upon to remember the key for the API 731. The key for the API 731 limits the access to libraries associated with the other server 730 which are also associated with the API. The key held by SAM device 740 will be used to limit the amount of information and programming that can be accessed by the server 702 or the system 700.

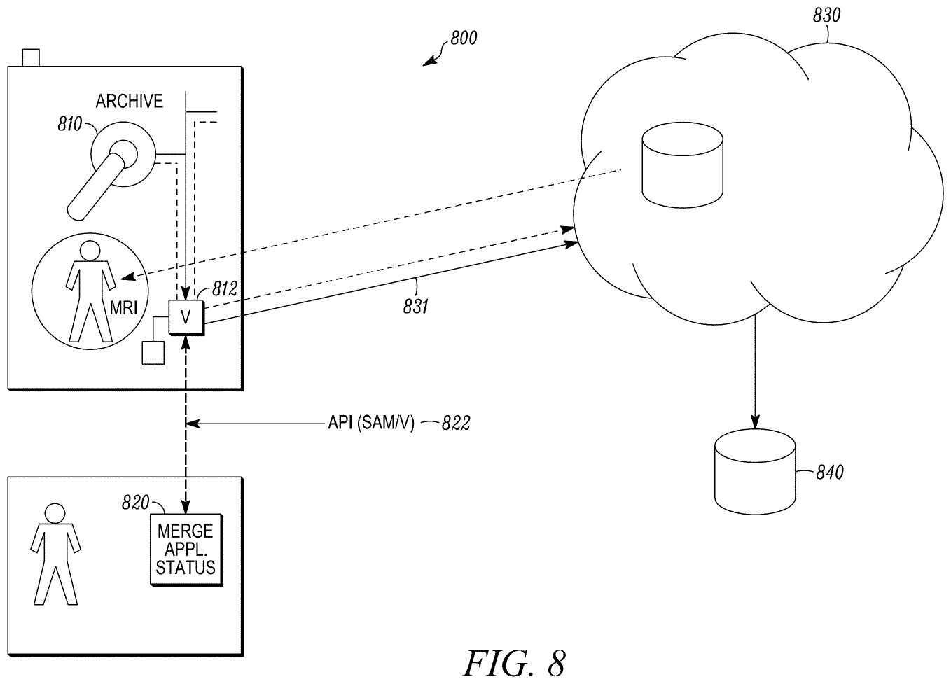

FIG. 8 is a schematic drawing of a computing system 800 having SAM 812 configured within a system 800, according to an example embodiment. The SAM device 812 is capable of manual sends as well as automatic sends of medical information. The system 800 includes a modality 810. The modality can be any imaging device or actually any source of patient health information. In this particular system 800 the institution has decided to automatically archive images or patient health information into a cloud 830. The system 800 includes a SAM device which automatically routes the medical data to the cloud 830. The SAM device 812 recalls the API needed to connect to the cloud. It should be noted that more than one modality may be on a system 800. The SAM device 812 will also know the destination for archiving images from other modalities. From the cloud 830, and more particularly from the archive on the cloud 830 others, such as another network 840, are able to access the data from the archive on the cloud 830. The system can also include an interface 820 for determining the status of the various sends and receives for medical information. The SAM device manages and API 822 which allows limited access to the system 800. The API 822 allows the module 820 for the interface to have access to enough data to produce the interface 820. An operator can also be allowed to resend certain records that a failed to go across the interconnection 831 to the cloud 830, for example. The status interface 820 shows the status of the sent or received medical information. If the sender receive has failed, the status screen or interface 820 can also include reasons for the failure. In addition to a status screen interface 820 there may be other interfaces provided by the SAM device.

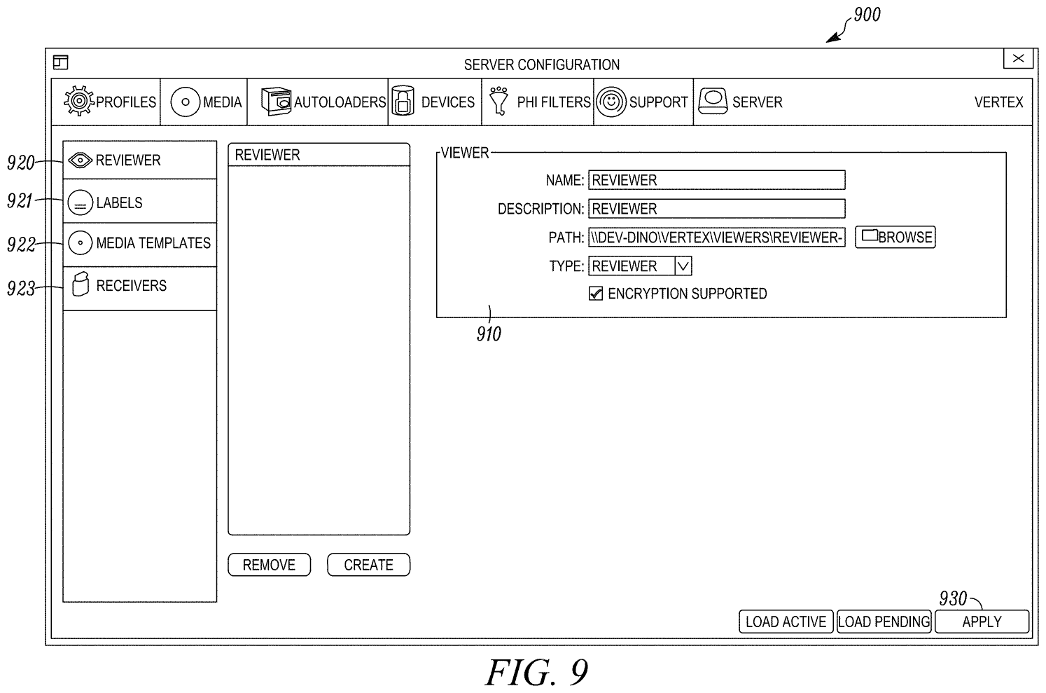

FIG. 9 is a screen shot of a SAM user interface 900 for configuring a server, according to an example embodiment. The user interface 900 for configuring the server, as shown in FIG. 9, shows a prompt or set of prompts 910 for selecting viewers needed to look at medical information on various media. This particular example the prompts 910 include the name of the viewing software, the description of the viewing software as well as the path to the reviewing software. Also included is an indication of whether encryption is supported. As can be seen, in addition to designating viewers there are categories for configuring labels 921, configuring media templates 922 and for configuring receivers 923. Once the appropriate configuration is selected, the configuration can be applied by activating or pressing the apply 930.

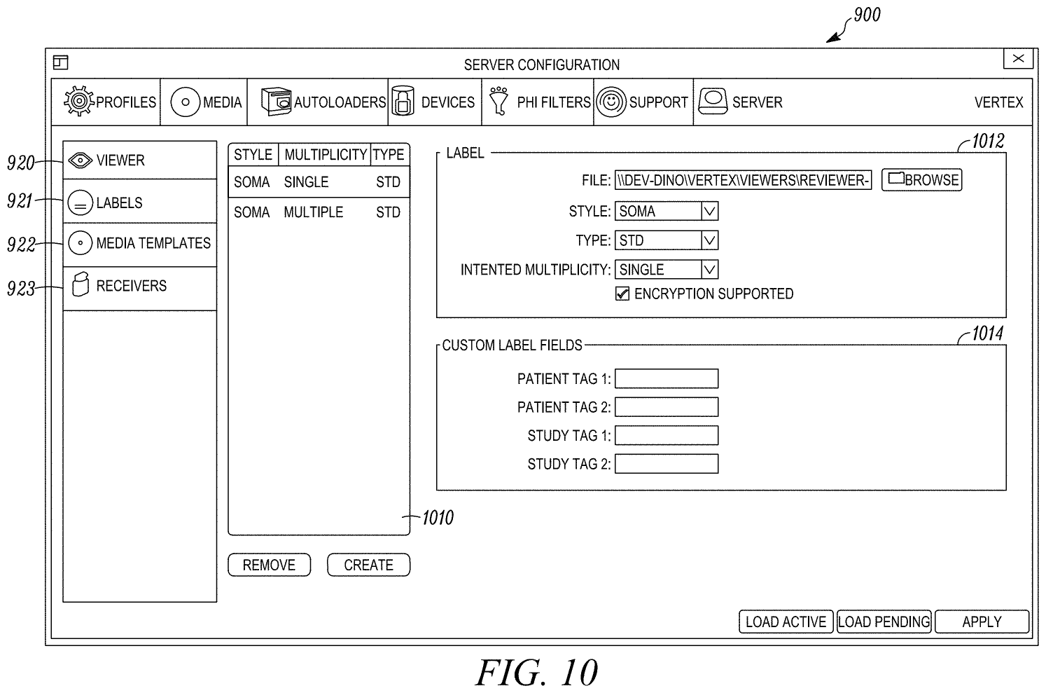

FIG. 10 is a screen shot of a SAM user interface 900 which is being used to define the label for a type of media, according to an example embodiment. The label button 921 has been enabled. A style box 1010 presents one of several styles of labels that can be selected. The style box, therefore prompts the user to select the style. Once the style selected, there are two prompts on the interface. One of the prompts 1012 as for the label. The file path to the label is designated as well as the style and type and intended multiplicity. The other of the prompts 1014 is for custom label and prompts the user to select certain custom label fields.

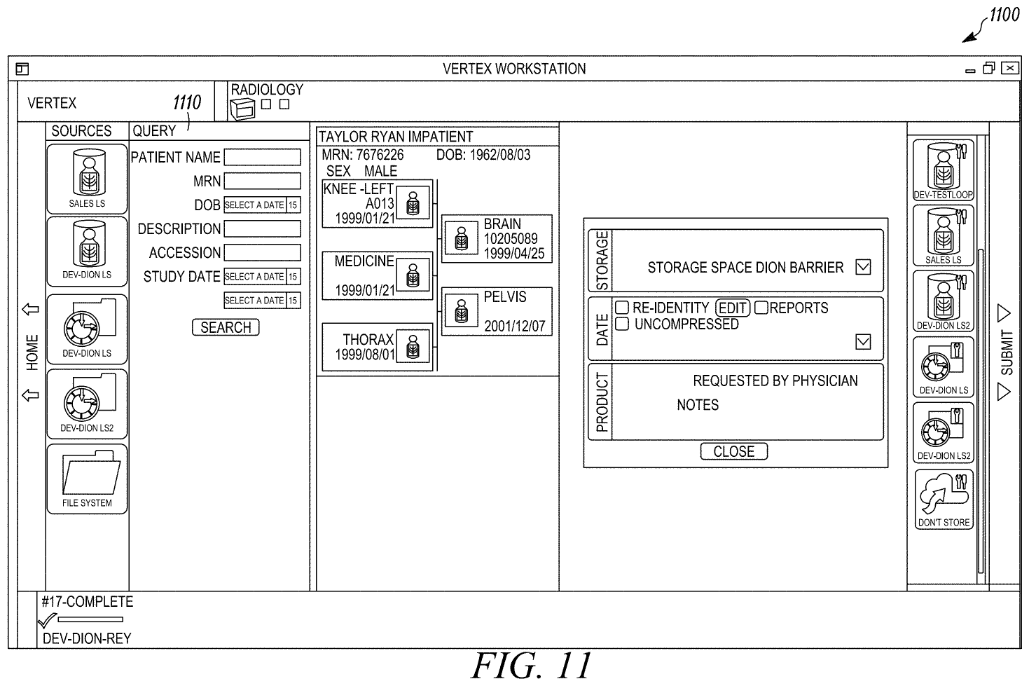

FIG. 11 is a screen shot of a SAM user interface 1100 which is used to define a query for a patient's health information, according to an example embodiment. The interface 1100 includes a query prompt 1110. The prompt 1110 includes fields or prompts for the patient's name and date of birth as well as for a study description. The steady description can be searched based upon an accession number or based on a study date or range of study dates. The query prompt also includes a search button to launch the query or the search after the prompts associated with the query prompt 1110 have been filled or partially filled. As shown in FIG. 11, the query has yielded several patient studies. In the alternative the prompt 1110 has yielded several types of medical information. Also included in the query screen 1100 is a send prompt 1120. The send prompt 1120 includes prompts for where to store the patient information, the data associated with the send request as well as products associated with the send request. In this particular send prompt 1120 notes from a medical professional or other associated person will also be sent. The screen shots shown here show certain aspects of the interface 900. Other aspects of the interface can be implemented through the user interface. Many of the aspects are discussed above.

The user interface 900 employees drag-and-drop functionality to make completing tasks or configuring a system easier on the user. Using the SAM device, data profiles and data profile requests can be easily modified and made. In addition, a system can be configured for automatic or user-initiated sharing of medical information. The SAM device is capable of seamless integration with existing hardware. DICOM and many other image and document files for handling patient information can be handled with ease. Medical information can be sent to another location via secure DICOM share or can be transported to a cloud for storage or for transport to another user.

In some embodiments, a computer system for medical information includes a source of medical information. The medical information includes at least one DICOM files. The computer system also includes a memory for storing medical information, including DICOM files, from the source, and a processor communicatively coupled to the memory and the source, and a module executable on the processor including instructions for receiving medical information from the source, storing information, and automatically delivering the medical information to a plurality of destinations substantially simultaneously. Substantially simultaneously as used herein can refer to when a plurality of instances of data can be sent to one destination or a plurality of destinations at the same time, at the same time and about the same time. The computer system is also is communicatively coupled to a data base that includes connection information for: a plurality of clouds and associates each of the clouds to the plurality of destinations, and at least one destination for receiving medical information directly from the computer system, wherein a destination is designated and the database provides the module with the connection information needed to securely send the information to the destination. In one embodiment, the module operates to store medical information at a cloud location to enable an automatic mode for storing the medical information.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment. These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A computer system for medical information comprising:
a polarity of sources of medical information, at least one source of medical information, including DICOM files;
a memory for storing medical information, including DICOM files, from the plurality of sources;
a processor communicatively coupled to the memory and the plurality of sources;
a module for handling information, the module executable on the processor including a set of instructions for receiving medical information from the plurality of sources, storing information, and automatically delivering the medical information to a plurality of destinations;
a database that includes connection information for the plurality of destinations that include:
a plurality of clouds, the data base associating at least one cloud to at least one of the plurality of destinations, and
at least one destination for receiving medical information directly from the computer system, wherein a destination is designated, and the database provides the module with the connection information needed to securely send the information to the destination.

2. The computer system of claim 1 wherein the database tracks referral sources and the referral sources method of connectivity such as one of the plurality of cloud providers, a portable storage device, a media device or a direct secure share.

3. The computer system of claim 1 wherein the medical information includes at least one of lab results, medical professionals notes, and lab report.

4. The computer system of claim 1 wherein the medical information includes information generated by other medical devices.

5. The computer system of claim 1 wherein the module parses incoming medical information to get physician, patient and study information.

6. The computer system of claim 5 wherein the module can modify the format of a report to one of a plurality of formats.

7. The computer system of claim 5 wherein the module relates pieces of medical information to one another using an Accession number.

8. The computer system of claim 5 wherein the module relates pieces of medical information to one another using best data available.

9. The computer system of claim 8 wherein the module performs a query of an archive to find archived information related to the medical information.

10. The computer system of claim 9 the module is configurable to search selected archives.

11. The computer system of claim 9 the module is configurable to search archives for selected time frames to find relevant prior studies.

12. The computer system of claim 9 the module is configurable to exclude selected series from a study.

13. The computer system of claim 1 wherein selected tasks are done as part of an automated operation.

14. The computer system of claim 1 wherein the module presents a drag and drop user interface that presents prompts related to sending information from the computer system to another destination.

15. The computer system of claim 1 wherein the module further comprises hardware components and software components.

16. A computerized method for retrieving information from a computer system comprising:
presenting a set of prompts to define a query for medical information;
receiving inputs regarding medical information to be retrieved;
presenting a set of prompts for defining which of the inputs regarding the medical information to send;
presenting another set of prompts for determining a plurality of destinations for the medical information;
receiving inputs regarding the medical information to send, and the plurality of destinations of the medical information;
determining connection information needed to send the medical information to each of the plurality of destinations, at least one of the destinations including a cloud;
determining if additional information is needed to send the medical information, the additional information associated with each of the plurality of destinations; and
securely sending the medical information to the plurality of destinations at about the same time by designating the plurality of destinations and obtaining connection information from a memory location.

17. The computerized method of claim 16 wherein determining if additional information is needed to send the medical information includes providing a key for an application programming interface (API).

18. A computerized method for receiving information from a computer system comprising:
receiving medical information from a network;
parsing the medical information received;
determining a plurality of destinations for the received information;
determining connection information needed to send the medical information to each of the plurality of destinations, at least one of the destinations including a cloud; and
determining if additional information is needed to send the medical information, the additional information associated with each of the plurality of destinations; and
securely sending the medical information to the determined plurality of destinations by designating the plurality of destinations without designating the connection information, the connection information obtained from a memory location.

19. The computerized method of claim 18 wherein sending the medical information to one or more of the determined plurality of destinations includes sending the medical information to a cloud for storage at one or more cloud storage locations.

20. A computer system for medical information comprising:
a source of medical information, including DICOM files;
a memory for storing medical information, including DICOM files, from the source;
a processor communicatively coupled to the memory and the source;
a module executable on the processor including instructions for receiving medical information from the source, storing information, and automatically delivering the medical information to a plurality of destinations substantially simultaneously;
a database that includes connection information for:
a plurality of clouds and associates each of the clouds to the plurality of destinations, and
at least one destination for receiving medical information directly from the computer system, wherein a destination is designated and the database provides the module with the connection information needed to securely send the information to the destination.
